(19) United States
(12) Patent Application Publication
(10) Pub. No.: US 2019/0233810 A1
(43) Pub. Date: Aug. 1, 2019

(54) Method for Isolating Nucleic Acids with Bivalent Cations and Elution with a Cation Chelating Agent

(71) Applicants: LGC Genomics Limited, Hoddesdon (GB); LGC Genomics GmbH, Berlin (DE)

(72) Inventors: Frank Ullrich Schubert, Berlin (DE); Scott David Goodwin, Dunmow (GB)

(73) Assignees: LGC Genomics Limited, Hoddesdon (GB); LGC Genomics GmbH, Berlin (US)

(21) Appl. No.: 16/312,212

(22) PCT Filed: Jun. 30, 2017
(86) PCT No.: PCT/GB2017/051930

§ 371 Date: Dec. 20, 2018

(30) Foreign Application Priority Data
Jun. 30, 2016 (GB) 1611425.8

(51) Int. Cl. C12N 15/10

(52) U.S. Cl. CPC C12N 15/1013

(57) ABSTRACT

The present invention relates to improved methods of isolating nucleic acids. In particular the method comprises the use of a wash buffer comprising bivalent cations prior to elution of the nucleic acid.

The present invention relates to improved methods of isolating nucleic acids. In particular the method comprises the use of a wash buffer comprising bivalent cations prior to elution of the nucleic acid.

Since the establishment of nucleic acid analytical techniques in clinical and molecular biology laboratory practice, methods relating to nucleic acid purification have undergone rapid technological development. In particular, much focus has been given to improving nucleic acid yield and purity while also streamlining the process and/or making it applicable to automation (e.g. using liquid handling systems).

Solid phase extraction methods of nucleic acid isolation are commonly used. In such methods, nucleic acids in a sample are adsorbed to solid surfaces, washed to remove impurities, and then eluted from the solid phase in a solution. One of the earliest examples of methods utilising that principle, is described in Gillespie and Vogelstein, 1979 (Proc. Natl. Acad. Sci. USA, 76: 615-619) and uses siliceous materials, such as finely divided glass, to adsorb nucleic acids and buffer solutions containing substantially chaotropic salts. Similarly, Boom et al, 1999 (J. Clin. Microbiol., 37: 615-619) also describes a method which utilises siliceous matrices and chaotropic buffer systems to adsorb and isolate DNA from complex biological samples such as cerebrospinal fluid and urine. One disadvantage of such methods of nucleic acid isolation is that the nucleic acid only remains adsorbed to the solid surfaces under the chaotropic conditions provided by the binding buffer. In addition, chaotropic agents that remain associated with the isolated nucleic add can interfere with sensitive downstream applications (e.g. enzymatic reactions, nucleic acid analysis, etc.)

Removal of chaotropic salts from the nucleic acid and solid surfaces is generally done using wash solutions containing a significant proportion of water-miscible organic solvents (usually >50% by volume). However, those organic solvents can also inhibit sensitive downstream processes in a similar way to the chaotropic agents they are intended to remove. Therefore, prior to elution of the adsorbed nucleic acid, the organic solvents must be removed to minimise the potential for inhibition of downstream processes, but doing so considerably slows down and adds complexity to the process.

Methods based on the use of non-chaotropic salts are described in DE 19856064 and US 2001041332. In those methods so-called kosmotropic salts are used to promote binding of nucleic acids to silicate surfaces, although the inconveniences associated with the removal of organic solvents from the system are still present.

In contrast, WO 9609379 describes the use of magnetic microparticles carrying negatively charged carboxyl groups and buffer systems with a high proportion of polyethylene glycol (i.e. not chaotropic salts) for the isolation of DNA. As with the chaotropic and kosmotropic agent-based methods, the use of organic solvents in wash buffers is required to remove unwanted components of the binding buffer. A similar approach is described in WO 02066993, where cellulose derivatives with magnetic properties are used in conjunction with polyethylene glycol-containing buffers to adsorb nucleic acid to the solid surfaces.

The use of silicate materials in combination with buffer systems containing organic solvents, is described in EP 0512767 and in those systems the polarity ratios of the components play a crucial role in the method. Again, organic solvents are present in the wash buffers and must be removed by evaporation prior to elution of the adsorbed nucleic acids, otherwise subsequent analysis/use of the purified nucleic acids may be compromised.

A fundamentally different type method, which uses the so-called charge switch mechanism (e.g. CST® from Invitrogen), is described in WO 0248164. In that method an ion exchange system is used to bind nucleic acids to positively charged surfaces in an aqueous buffer system with a weakly acidic pH. The weakly acidic pH is maintained and the contaminants are then washed from the system, before the bound nucleic acid is eluted using weakly alkaline conditions. Such a method is made possible by the introduction into the system of ion exchange active groups (e.g. aliphatic or heterocyclic amino groups) whose net charge is positive or neutral, depending on the pH of the medium. The binding of nucleic acids to the solid surface occurs due to the interaction of the negatively charged nucleic acids to the positively charged surface of the particles.

One problem with ion exchange-based systems is that because the isolation of the nucleic acid is based solely on charge, any other macromolecules with a net negative charge (under the conditions used in the method) are also likely to be purified along with the nucleic acids. Potential contaminants include proteins and polysacchardes. In addition, ion exchange interactions are very sensitive to high ionic strength and the presence of detergents in the binding buffer, and therefore require a very accurate setting of the binding conditions. In addition to pH, factors such as salt concentration and, in particular, the amount of cationic detergents need to be carefully considered. Those considerations can lead to problematic compromises to the design of lysis conditions for the digestion of the initial sample material.

WO2010018200 A1 and DE102007009347 B4 describe the use of oligomeric amines in order to keep nucleic acids adsorbed to surfaces bearing weak organic acid groups. Those oligomeric amines are relatively expensive and, even when used in very low concentrations, have toxic properties. Surprisingly, it was found that using bivalent cations instead of oligomeric amines, particularly bivalent cations of alkaline earth metals, can keep nucleic acids adsorbed to a weak organic acid-bearing binding surface with the same efficiency. It is known in the art that magnesium ions interact with nucleic acids and promote stabilisation of those molecules. Indeed, magnesium ions are important co-factors in a number of nucleic acid-based applications (e.g. Polymerase Chain Reaction (PCR)). In contrast, calcium ions, despite being bivalent cations in the same group as magnesium, behave differently in their interactions with nucleic acids. In fact, calcium ions have been shown to be inhibitors of nucleic acid-related processes such as PCR (Schrader et al. (2012) J. Appl. Microbiol., 113: 1014-1026). Thus, the ability of calcium ions to drive the binding of nucleic acids to surfaces bearing weak organic acid groups was surprising. Furthermore, it was also found that complexing bivalent cations (such as calcium ions) using chelating agents and/or increasing the pH of the binding surface to alkaline conditions can break the interaction between the nucleic acids and the binding surface releasing the nucleic acids back into the liquid phase for elution. In methods known in the art, elution of nucleic acid is generally mediated by the addition water or very weakly alkaline solutions.

In a first aspect the invention provides methods of isolating a nucleic acid comprising the steps of:
(a) providing a binding mixture comprising nucleic acid;
(b) contacting the binding mixture with a polar solid support such that nucleic acid adsorbs to the surface of the solid support;
(c) removing unbound binding mixture;
(d) washing the solid support with a wash buffer;
(e) adding an elution buffer and removing nucleic acid from the solid support.

By “isolating a nucleic acid” we include the meaning of substantially purifying nucleic acid from a given sample. Samples from which nucleic acid may be purified include, but are not limited to, eukaryotic and prokaryotic cells, clinical samples such as tissue samples or blood samples, laboratory reaction mixtures such as PCR or restriction digest reactions, forensic samples including those obtained from a crime scene (e.g. physical evidence, blood, saliva, etc.), soil samples, and plant material such as leaves, seeds, or roots.

By “binding mixture” we include the meaning of a solution comprising a sample comprising nucleic acid and a binding buffer.

By “binding buffer” we include the meaning of a buffer solution which has a composition that produces conditions which promote adsorption of nucleic acid to the solid support.

By “elution buffer” we include the meaning of a buffer solution that acts to release the adsorbed nucleic acid from the solid support.

In preferred embodiments the wash buffer comprises bivalent cations in aqueous solution. In other embodiments the bivalent cations are from alkaline earth metals from Group II of the periodic table. In further embodiments the bivalent cations are in the form of their chlorides. In preferred embodiments the bivalent cations are calcium or barium ions. In a more preferred embodiment the bivalent cations are calcium ions.

In other embodiments the wash buffer is an aqueous solution in pure water or an aqueous buffer solution. In further embodiments the aqueous buffer solution has a pH value between pH 4.0 and pH 7.0. In a preferred embodiment the aqueous buffer has a pH value between pH 6.0 and pH 6.5.

Aqueous buffer solutions suitable for use in the methods of the invention are well known to the skilled person and include, for example, solutions of Tris (2-Amino-2-(hydroxymethyl)-propan-1,3-diol) and Bis-Tris (Bis(2-hydroxymethyl)amino-tris(hydroxymethyl)methane).

In preferred embodiments the wash buffer comprises bivalent cations at a concentration between 0.1 mM and 10 mM, 0.1 mM and 9 mM, 0.1 mM and 8 mM, 0.1 mM and 7 mM, 0.1 mM and 6 mM, 0.1 mM and 5 mM, 0.2 mM and 4.5 mM, 0.3 mM and 4 mM, 0.4 mM and 3.5 mM, 0.5 mM and 3 mM, 0.6 mM and 2.5 mM, 0.7 mM and 2 mM, 0.8 mM and 1.5 mM, or 0.9 mM and 1 mM. In a more preferred embodiment the wash buffer comprises bivalent cations at a concentration between 1 mM and 5 mM.

In some embodiments the nucleic acid is removed from the surface of the solid support by complexing bivalent cations.

By “complexing” we include the meaning of making an atom or molecule form an association with another atom or molecule, i.e. by forming a complex.

In further embodiments the elution buffer comprises a chelating agent.

By “chelating agent” we include the meaning of a substance whose molecules can form several bonds to a single metal ion.

In some embodiments, the chelating agent complexes the bivalent cations provided in the wash buffer, promoting release of the nucleic acid from the surface of the solid support.

In some embodiments the chelating agent is selected from EGTA, EDTA, EDDS, MGDA, IDS, polyaspartic acid, GLDA, BAPTA, and citric acid. In preferred embodiments the chelating agent is EGTA.

In preferred embodiments the elution buffer comprises a chelating agent at a concentration between 0.1 mM and 5 mM, 0.1 mM and 4.5 mM, 0.1 mM and 4 mM, 0.1 mM and 3.5 mM, 0.1 mM and 3 mM, 0.1 mM and 2.5 mM, 0.1 mM and 2 mM, 0.1 mM and 1.5 mM, 0.1 mM and 1 mM, 0.2 mM and 0.9 mM, 0.3 mM and 0.8 mM, 0.4 mM and 0.7 mM, 0.5 mM and 0.6 mM, preferably at a concentration between 0.1 mM and 1 mM.

In some embodiments the elution buffer comprising a chelating agent has a pH value between pH 7.0 and pH 10.0, pH 7.0 and pH 9.5, pH 7.0 and pH 9.0, pH 7.0 and pH 8.5, pH 7.0 and pH 8.0, pH 7.0 and pH 7.5. In a preferred embodiment the elution buffer comprising a chelating agent has a pH value between pH 7.0 and pH 9.0.

In other embodiments the nucleic acid is removed from the surface of the solid support by raising the pH of the solid support to alkaline conditions.

By “alkaline conditions” we include the meaning of any condition with a pH value greater than pH 7.0.

In some embodiments the elution buffer has a pH value between pH 7.1 and pH 10.0. In a preferred embodiment, the elution buffer has a pH value between pH 8.0 and pH 10.0.

In a most preferred embodiment the elution buffer has a pH value between pH 9.0 and pH 10.0.

The pH of the elution buffers disclosed herein can be supported by additional buffering substances. Additional buffering substances suitable for use in the methods of the invention are well known the skilled person and include, for example, Tris, aminomethyl propanol (AMP), and citrate.

In some embodiments the nucleic acid is removed from the surface of the solid support by complexing bivalent cations as described above, and/or by raising the pH of the solid support to alkaline conditions as described above, and by raising the temperature of the solid support. In some embodiments the temperature of the solid support is raised to at least 30° C. In some embodiments the temperature of the solid support is raised to between 30° C. and 75° C., 35° C. and 70° C., 40° C. and 65° C., 45° C. and 60° C., 40° C. and 50° C., or 45° C. and 55° C. In a preferred embodiment the temperature of the solid support is raised to between 30° C. and 75° C.

In some embodiments the surface of the solid support binds bivalent cations. In preferred embodiments, the binding of bivalent cations to the surface of the solid support leads to adsorption of nucleic acid to the surface of the solid support.

In some embodiments the surface of the solid support comprises weak organic acids.

By “weak organic acids” we include the meaning of an organic compound with acidic properties. Non-limiting examples of weak organic acids include formic acid, malic acid, maleic acid, acetic acid, propionic acid, butyric acid, valeric acid, caproic acid, oxalic acid, lactic acid, citric acid, and benzoic acid.

In further embodiments the weak organic acids are selected from the list consisting of phosphonic acids, aliphatic carboxylic acids, and aromatic carboxylic acids.

In preferred embodiments the weak organic acids are homo- or hetero-polymers.

By “homo-polymer” we mean a polymer comprising a single species of monomer.

By “hetero-polymer” we mean a polymer comprising two or more different species of monomer.

In further embodiments the weak organic acid polymer is selected from the list consisting of poly-acrylic acid, poly-phosphonic acid, poly-methacrylic acid, poly-maleic acid, a hetero-polymer of acrylic acid and methacrylic acid, a hetero-polymer of methacrylic acid and maleic acid, and a hetero-polymer of acrylic acid, methacrylic acid and maleic acid.

In other embodiments the binding mixture comprises a binding buffer. In further embodiments the binding buffer comprises an organic solvent that is miscible with water, and/or a chaotropic agent, and/or a detergent.

By “organic solvent that is miscible with water” we include the meaning of an organic substance that is capable of dissolving a solute and is capable of forming a homogeneous mixture with water. Non-limiting examples of water-miscible organic solvents include ethanol, methanol, 1-propanol, propan-2-ol, acetone, acetonitrile, 1,2-butanediol, 1,3-butanediol, 1,4-butanediol, 2-butoxyethanol, dimethytformamide, dimethoxyethane, dimethyl sulfoxide, 1,4-dioxane, ethylene glycol, furfuryl alcohol, glycerol, 1,3-propanediol, 1,5-pentanediol, propanoic acid, propylene glycol, pyridine, tetrahydrofuran, and triethylene glycol.

By “chaotropic agent” we mean a substance in a water solution that can disrupt the hydrogen bonding network between water molecules. Non-limiting examples of chaotropic agents include guanidinium hydrochloride, urea, thiourea, lithium perchlorate, and lithium acetate.

By “detergent” we include the meaning of an ionic or non-ionic surfactant or mixture of surfactants that are not inactivated by hard water and have wetting-agent and/or emulsifying-agent properties.

In some embodiments volume/volume percentage of organic solvent in the binding buffer is at least 5%. In a preferred embodiment the volume/volume percentage of organic solvent in the binding buffer is between 5% and 50%. In a preferred embodiment the volume/volume percentage of organic solvent in the binding buffer is between 10% and 50%. In a more preferred embodiment the volume/volume percentage of organic solvent in the binding buffer is between 20% and 50%. In a further preferred embodiment the volume/volume percentage of organic solvent in the binding buffer is between 30% and 50%. In a most preferred embodiment the volume/volume percentage of organic solvent in the binding buffer is between 40% and 50%.

In other embodiments the concentration of chaotropic agent in the binding buffer is at least 0.5M. In a preferred embodiment the concentration of chaotropic agent in the binding buffer is between 0.5M and 3M, 0.5M and 2.75M, 0.5M and 2.5M, 0.5M and 2.25M, 0.5M and 2M, 0.5M and 1.75M, 0.5M and 1.5M, 0.5M and 1.25M, 0.5M and 1 M, 0.6M and 1.4M, 0.7M and 1.3M, 0.8M and 1.2M, or 0.9M and 1.1M. In a most preferred embodiment the concentration of chaotropic agent in the binding buffer is between 0.5M and 1.5M.

In some embodiments the volume/volume percentage of detergent in the binding buffer is at least 0.5%. In a preferred embodiment the volume/volume percentage of detergent in the binding buffer is between 5% and 20%. In another preferred embodiment the volume/volume percentage of detergent in the binding buffer is between 7% and 15%. In a most preferred embodiment the volume/volume percentage of detergent in the binding buffer is between 8% and 12%.

In certain embodiments the organic solvent in the binding buffer is an alcohol. In a preferred embodiment the alcohol is a low molecular weight alcohol. Low molecular weight alcohols suitable for use in the methods of the invention are well known to the skilled person and include, for example, ethanol, 1-propanol, or propan-2-ol.

In certain embodiments the chaotropic agent in the binding buffer is a guanidinium salt. Preferred guanidinium salts are guanidinium thiocyanate and guanidinium hydrochloride. In a preferred embodiment the guanidinium salt is guanidinium hydrochloride.

In certain embodiments the detergent in the binding buffer is an ionic or non-ionic detergent. Preferred non-ionic detergents are polyethylene glycol (PEG)-based detergents such as, for example, Tween-20 or Tween-80. Preferred ionic detergents are detergents comprising weak organic acid groups, for example, sodium lauroyl sarcosinate.

In some embodiments the solid support comprises microparticles. In certain embodiments the microparticles have superparamagnetic properties.

In other embodiments the microparticles have a diameter of at least 1 μm. In a preferred embodiment the microparticles have a diameter between 1 μm and 50 μm. In a most preferred embodiment the microparticles have a diameter between 1 μm and 20 μm.

In further embodiments the isolated nucleic acid is DNA, RNA, PNA, GNA, TNA, or LNA. In preferred embodiments the isolated nucleic acid is DNA or RNA.

In other embodiments the isolated nucleic acid is at least 20 nucleotides in length.

In certain embodiments the nucleic acid is isolated from a starting sample.

By “starting sample” we include the meaning of any sample from which nucleic acid may be obtained.

In other embodiments, prior to isolation of nucleic acid the starting sample is subjected to one or more of: chemical treatment, enzymatic treatment, and/or mechanical treatment.

Examples of chemical treatment include, but are not limited to treatment with detergents, or treatment with cell wall degrading agents.

Examples of enzymatic treatment include, but are not limited to treatment with proteases, treatment with cellulases, or treatment with amylases.

Examples of mechanical treatment include, but are not limited to grinding, milling, crushing, treatment with ultrasound, or generally applying mechanical stress to a sample.

In some embodiments the starting sample comprises laboratory contaminants. In certain embodiments the laboratory contaminants comprise Polymerase Chain Reaction (PCR) reagents, restriction enzyme digest reagents, in vitro reagent systems for modifying and/or processing nucleic acids, acrylamide gel, or agarose gel.

In other embodiments the starting sample comprises biological material. In certain embodiments the biological material comprises eukaryotic or prokaryotic cells. In further embodiments the cells are animal cells, plant cells, fungal cells, bacterial cells, archaeal cells, or protozoan cells.

In some embodiments the biological material is a bodily fluid or solid biological material from an animal. In certain embodiments the bodily fluid or solid biological material from an animal is blood, plasma, serum, urine, faeces, saliva, semen, nail, hair, or tissue.

In a particular embodiment the starting sample is material obtained for forensic analysis. In a further embodiment the material obtained for forensic analysis comprises saliva, blood, urine, faeces, semen, sweat, tears, hair, nail, or any tissue.

In other embodiments the nucleic acd remains adsorbed to the surface of the solid support even when the chemical conditions which promote adsorption to the surface of the solid support are no longer present.

In some embodiments the elution buffer is an aqueous elution buffer. Preferred aqueous elution buffers comprise tris(hydroxymethyl)aminomethane at a concentration between 1 mM and 50 mM. In a preferred embodiment the aqueous elution buffer comprises tris(hydroxymethyl)aminomethane at a concentration between 1 mM and 20 mM. In a more preferred embodiment the aqueous elution buffer comprises tris(hydroxymethyl)aminomethane at a concentration between 5 mM and 15 mM. In a most is preferred embodiment the aqueous elution buffer comprises tris(hydroxymethyl)aminomethane at a concentration of 10 mM.

Other buffering substances suitable for use in the methods of the invention are those that have buffering capacity between a pH range of pH 7.0 and pH 10.0. Such buffering substances are well known to the skilled person and include, for example, aminomethyl propanol (AMP).

Any elution buffer of the present invention may also comprise a preservative or chelating agent.

Preservatives suitable for use in the methods of the invention are well known to the skilled person and include, for example, sodium azide. In certain embodiments the elution buffer comprises sodium azide at a volume/volume concentration of 1% or less.

Chelating agents suitable for use in the methods of the invention are well known to the skilled person and include, for example, EDTA. In certain embodiments the elution buffer comprises EDTA at a concentration of 1 mM or less.

In a second aspect the invention provides a kit for isolating nucleic acid wherein the kit comprises:
(a) a polar solid support as defined in any of the embodiments of the first aspect of the invention;
(b) a binding buffer as defined in any of the embodiments of the first aspect of the invention;
(c) a wash buffer as defined in any of the embodiments of the first aspect of the invention; and,
(d) instructions for use.

In some embodiments the kit further comprises:
(e) an elution buffer as defined in any of the embodiments of the first aspect of the invention.

The listing or discussion of an apparently prior-published document in this specification should not necessarily be taken as an acknowledgement that the document is part of the state of the art or is common general knowledge.

The present invention will now be described in more detail with reference to the following non-limiting figures and examples.

### DESCRIPTION OF THE FIGURES

FIG. 1: Isolation of nucleic acid from a salmon sperm DNA solution. DNA was isolated from salmon sperm DNA solution using the method described in Example 1 and resolved by 0.8% agarose gel electrophoresis.
(A) Lambda DNA (200 ng in 8 μL).
(B) DNA isolated from salmon sperm DNA (Example 1: 5 mM CaCl₂ wash) (8 μL eluate).

FIG. 2: Isolation of DNA from plant leaf material from parsley. DNA was isolated from plant material from parsley using the Sbeadex Maxi Plant DNA extraction kit (LGC Genomics GmbH, Cat. No. 41602/41620) or the methods described in Examples 2-4.
(A) Lambda DNA (200 ng in 8 μL).
(B) DNA isolated from parsley leaf (Sbeadex Maxi Plant Kit) (8 μL eluate).
(C) DNA isolated from parsley leaf (Example 2: 5 mM CaCl₂ wash) (8 μL eluate).
(D) DNA isolated from parsley leaf (Example 3: 1 mM CaCl₂ wash) (8 μL eluate).
(E) DNA isolated from parsley leaf (Example 4: 0.1 mM CaCl₂ wash) (8 μL eluate).

FIG. 3: Isolation of DNA from plant leaf material from soy. DNA was isolated from plant material from soy using the method described in Example 5. Each of lanes R1-R8 represents a replicate DNA isolation experiment (8 μL eluate per well).

FIG. 4: Isolation of DNA from plant leaf material from sunflower. DNA was isolated from plant material from sunflower using the method described in Example 6. Each of lanes R1-R8 represents a replicate DNA isolation experiment (8 μL eluate per well).

### EXAMPLE 1

This example relates to isolation of nucleic acid from a solution of salmon sperm DNA.

Commercially available DNA from salmon sperm (Sigma, Cat. No. 31149) was dissolved at a final concentration of 500 ng/μL in a buffer containing 1% cetyltrimethylammonium bromide (CTAB), 50 mM TrisHCl (pH 8), 2 mM EDTA and 2% (w/v) polyvinyl-pyrrolidon. A 200 μL aliquot of that DNA solution was mixed with 400 μL binding buffer PN (2.25M guanidinium hydrochloride, 15% Tween-20, 50% 1-propanol) and 10 μL of standard Sbeadex bead solution, as available in, for example, Sbeadex Maxi Plant DNA extraction kit (LGC Genomics GmbH, Cat. No. 41602/41620). The resulting mixture was shaken for 5 minutes at room temperature, at 100 rpm, keeping Sbeadex beads evenly suspended throughout the solution. Following shaking the Sbeadex beads were removed by application of a permanent magnet and the supernatant was removed.

The Sbeadex beads were resuspended in 400 μL wash buffer PN1 (1.5M guanidinium hydrochloride, 10% Tween-20, 20% 1-propanol) and shaken for 5 minutes. The Sbeadex beads were collected by application of a permanent magnet and the supernatant was removed. The Sbeadex beads were then resuspended in 400 μL of a wash buffer containing 5 mM calcium chloride in 10 mM TrisHCl (pH 8.0) and shaken for 5 minutes at room temperature, at 100 rpm, keeping the Sbeadex beads evenly suspended throughout the solution. As before, the Sbeadex beads were collected by application of a permanent magnet.

DNA bound to the Sbeadex beads was eluted by addition of 100 μL elution buffer containing 40 mM 2-aminomethylpropanol-2 (pH 10.0) and 0.1 mM ethylene glycol-bis(2-aminoethylether)-N,N,N′,N′-tetraacetic acid (EGTA). Elution was carried out at 50° C. with occasionally shaking of the tube to keep the Sbeadex beads in suspension.

The eluted DNA was then measured by UV spectrophotometry and the following readings were recorded:

| A_{260/280} | A_{260/230} | DNA concentration |
|---|---|---|
| ratio | Ratio | (ng/μL) |
| 2.0 | 3.6 | 17.0 |

An 8 μL sample of the purified DNA was resolved by 0.8% agarose gel electrophoresis, along with a known quantity of Lambda DNA, and stained with ethidium bromide (FIG. 1).

### EXAMPLE 2

This example relates to isolation of DNA from plant leaf material from parsley.

A sample of 8 g fresh parsley leaves was ground in 40 mL lysis buffer PN (2.25M guanidinium hydrochloride, 15% Tween-20, 50% 1-propanol), as available in, for example, Sbeadex Maxi Plant Kit (LGC Genomics GmbH, Cat. No. 41602/41620) and Incubated for 30 minutes at 60° C., according to the protocol of that kit.

After that incubation, 200 μL lysate was mixed with 400 μL binding buffer PN (2.25M guanidinium hydrochloride, 15% Tween-20, 50% 1-propanol) and 10 μL standard Sbeadex beads solution and shaken for 5 minutes at room temperature at 100 rpm, keeping the Sbeadex beads evenly suspended throughout the solution. Following shaking the Sbeadex beads were removed by application of a permanent magnet and the supernatant was removed.

The Sbeadex beads were resuspended in 400 μL wash buffer PN1 (1.5M guanidinium hydrochloride, 10% Tween-20, 20% 1-propanol) and shaken for 5 minutes. The Sbeadex beads were collected by application of a permanent magnet and the supernatant was removed. The Sbeadex beads were then resuspended in 400 μL of a wash buffer containing 5 mM calcium chloride in 10 mM TrisHCl (pH 8.0) and shaken for 5 minutes at room temperature at 100 rpm, keeping the Sbeadex beads evenly suspended throughout the solution. As before, the Sbeadex beads were collected by application of a permanent magnet.

DNA bound to the Sbeadex beads was eluted by addition of 100 μL elution buffer containing 40 mM 2-aminomethylpropanol-2 (pH 10.0) and 0.1 mM ethylene glycol-bis(2-aminoethylether)-N,N,N′,N′-tetraacetic acid (EGTA). Elution was carried out at 50° C. with occasionally shaking of the tube to keep the Sbeadex beads in suspension.

The DNA Isolation experiment was carried out in duplicate and the eluted DNA was measured by UV spectrophotometry (see Table 1 below). An 8 μL sample of each eluate of isolated DNA was resolved by 0.8% agarose gel electrophoresis (FIG. 2, lane B). As a comparison, DNA was isolated from the same starting sample using the Sbeadex Maxi Plant DNA extraction kit (LGC Genomics GmbH, Cat No. 41602/41620) (FIG. 2, lane A).

### EXAMPLE 3

This example relates to isolation of DNA from plant leaf material from parsley.

A sample of 8 g fresh parsley leaves was ground in 40 mL lysis buffer PN (2.25M guanidinium hydrochloride, 15% Tween-20, 50% 1-propanol), as available in, for example, Sbeadex Maxi Plant Kit (LGC Genomics GmbH, Cat. No. 41602/41620) and Incubated for 30 minutes at 60° C., according to the protocol of that kit.

After that incubation, 200 μL lysate was mixed with 400 μL binding buffer PN (2.25M guanidinium hydrochloride, 15% Tween-20, 50% 1-propanol) and 10 μL standard Sbeadex beads solution and shaken for 5 minutes at room temperature, at 100 rpm, keeping Sbeadex beads evenly suspended throughout the solution. Following shaking the Sbeadex beads were removed by application of a permanent magnet and the supernatant was removed.

The Sbeadex beads were resuspended in 400 μL wash buffer PN1 (1.5M guanidinium hydrochloride, 10% Tween-20, 20% 1-propanol) and shaken for 5 minutes. The Sbeadex beads were collected by application of a permanent magnet and the supernatant was removed. The Sbeadex beads were then resuspended in 400 μL of a wash buffer containing 1 mM calcium chloride in 10 mM TrisHCl (pH 8.0) and shaken for 5 minutes at room temperature, at 100 rpm, keeping Sbeadex beads evenly suspended throughout the solution. As before, the Sbeadex beads were collected by application of a permanent magnet.

DNA bound to the Sbeadex beads was eluted by addition of 100 μL elution buffer containing 40 mM 2-aminomethylpropanol-2 (pH 10.0) and 0.1 mM ethylene glycol-bis(2-aminoethylether)-N,N,N′,N′-tetraacetic acid (EGTA). Elution was carried out at 50° C. with occasionally shaking of the tube to keep the Sbeadex beads in suspension.

The DNA isolation experiment was carried out in duplicate and the eluted DNA was measured by UV spectrophotometry (see Table 1 below). An 8 μL sample of each eluate of isolated DNA was resolved by 0.8% agarose gel electrophoresis (FIG. 2, lane C). As a comparison, DNA was isolated from the same starting sample using the Sbeadex Maxi Plant DNA extraction kit (LGC Genomics GmbH, Cat. No. 41602/41620) (FIG. 2, lane A).

### EXAMPLE 4

This example relates to isolation of DNA from plant leaf material from parsley.

A sample of 8 g fresh parsley leaves was ground in 40 mL lysis buffer PN (2.25M guanidinium hydrochloride, 15% Tween-20, 50% 1-propanol), as available in, for example, Sbeadex Maxi Plant Kit (LGC Genomics GmbH, Cat. No. 41602/41620) and incubated for 30 minutes at 60° C., according to the protocol of that kit.

After that incubation, 200 μL lysate was mixed with 400 μL binding buffer PN (2.25M guanidinium hydrochloride, 15% Tween-20, 50% 1-propanol) and 10 μL standard Sbeadex beads solution and shaken for 5 minutes at room temperature, at 100 rpm, keeping Sbeadex beads evenly suspended throughout the solution. Following shaking the Sbeadex beads were removed by application of a permanent magnet and the supernatant was removed.

The Sbeadex beads were resuspended in 400 μL wash buffer PN1 (1.5M guanidinium hydrochloride, 10% Tween-20, 20% 1-propanol) and shaken for 5 minutes. The Sbeadex beads were collected by application of a permanent magnet and the supernatant was removed. The Sbeadex beads were then resuspended in 400 μL of a wash buffer containing 0.1 mM calcium chloride in 10 mM TrisHCl (pH 8.0) and shaken for 5 minutes at room temperature, at 100 rpm, keeping Sbeadex beads evenly suspended throughout the solution. As before, the Sbeadex beads were collected by application of a permanent magnet.

DNA bound to the Sbeadex beads was eluted by addition of 100 μL elution buffer containing 40 mM 2-aminomethylpropanol-2 (pH 10.0) and 0.1 mM ethylene glycol-bis(2-aminoethylether)-N,N,N′,N′-tetraacetic acid (EGTA). Elution was carried out at 50° C. with occasionally shaking of the tube to keep the Sbeadex beads in suspension.

The DNA isolation experiment was carried out in duplicate and the eluted DNA was measured by UV spectrophotometry (Table 1).

TABLE 1
Eluate DNA concentration after washes with Ca²⁺-containing buffers.

| | Ca²⁺ | | | |
|---|---|---|---|---|
| DNA | concentration | | | |
| isolation | in wash buffer | A_{260/280} | A_{260/230} | DNA concentration |
| method | (mM) | ratio | ratio | (ng/μL) |
| Example 2 | 5 | 1.8 | 1.8 | 48.5 |
| | 5 | 1.8 | 1.9 | 48.2 |
| Example 3 | 1 | 1.8 | 1.7 | 40.5 |
| | 1 | 1.8 | 1.7 | 42.5 |
| Example 4 | 0.1 | 1.7 | 1.7 | 31.8 |
| | 0.1 | 1.7 | 1.6 | 33.7 |

An 8 μL sample of each eluate of Isolated DNA was resolved by 0.8% agarose gel electrophoresis (FIG. 2, lane D). As a comparison, DNA was isolated from the same starting sample using the Sbeadex Maxi Plant DNA extraction kit (LGC Genomics GmbH, Cat. No. 41602/41620) (FIG. 2, lane A).

### EXAMPLE 5

This example relates to isolation of DNA from plant leaf material from soy.

Four punches (6 mm in diameter) were taken from dried soy leaf material and ground in 400 μL lysis buffer PN (2.25M guanidinium hydrochloride, 15% Tween-20, 50% 1-propanol) using a ball-milling instrument (Genogrinder) for 1 minute at 1,750 strokes per second. The resultant sample was incubated for 30 minutes at 60° C., according to the protocol of the Sbeadex Maxi Plant Kit (LGC Genomics GmbH, Cat No. 41602/41620).

After that incubation, 200 μL lysate was mixed with 400 μL binding buffer PN (2.25M guanidinium hydrochloride, 15% Tween-20, 50% 1-propanol) and 10 μL standard Sbeadex beads solution and shaken for 5 minutes at room temperature, at 100 rpm, keeping Sbeadex beads evenly suspended throughout the solution. Following shaking the Sbeadex beads were removed by application of a permanent magnet and the supernatant was removed.

The Sbeadex beads were resuspended in 400 μL wash buffer PN1 (1.5M guanidinium hydrochloride, 10% Tween-20, 20% 1-propanol) and shaken for 5 minutes. The Sbeadex beads were collected by application of a permanent magnet and the supernatant was removed. The Sbeadex beads were then resuspended in 400 μL of a wash buffer containing 0.1 mM calcium chloride in 10 mM TrisHCl (pH 8.0) and shaken for 5 minutes at room temperature, at 100 rpm, keeping Sbeadex beads evenly suspended throughout the solution. As before, the Sbeadex beads were collected by application of a permanent magnet.

DNA bound to the Sbeadex beads was eluted by addition of 100 μL elution buffer containing 40 mM 2-aminomethylpropanol-2 (pH 10.0) and 0.1 mM ethylene glycol-bis(2-aminoethylether)-N,N,N′,N′-tetraacetic acid (EGTA). Elution was carried out at 50° C. with occasionally shaking of the tube to keep the Sbeadex beads in suspension.

The DNA isolation experiment was repeated a total of eight times and the eluted DNA was measured by UV spectrophotometry (Table 2).

TABLE 2
Eluate DNA concentration following DNA isolation (8 replicates)

| | | | DNA |
|---|---|---|---|
| | | | concentration |
| Replicate number | A_{260/280} ratio | A_{260/230} ratio | (ng/μL) |
| R1 | 1.6 | 0.5 | 16.7 |
| R2 | 1.6 | 0.5 | 17.2 |
| R3 | 1.6 | 0.5 | 37.4 |
| R4 | 1.6 | 0.5 | 21.9 |
| R5 | 1.7 | 0.6 | 39.5 |
| R6 | 1.6 | 0.5 | 43.0 |
| R7 | 1.6 | 0.5 | 23.1 |
| R8 | 1.6 | 0.6 | 13.6 |

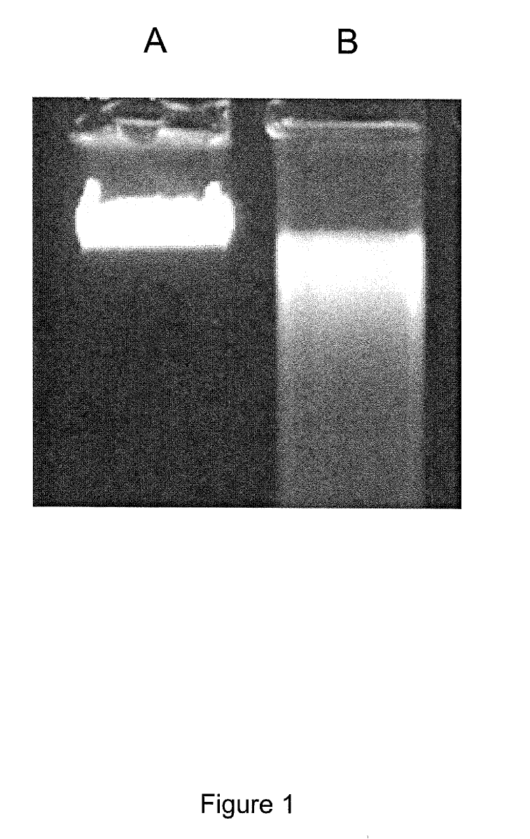

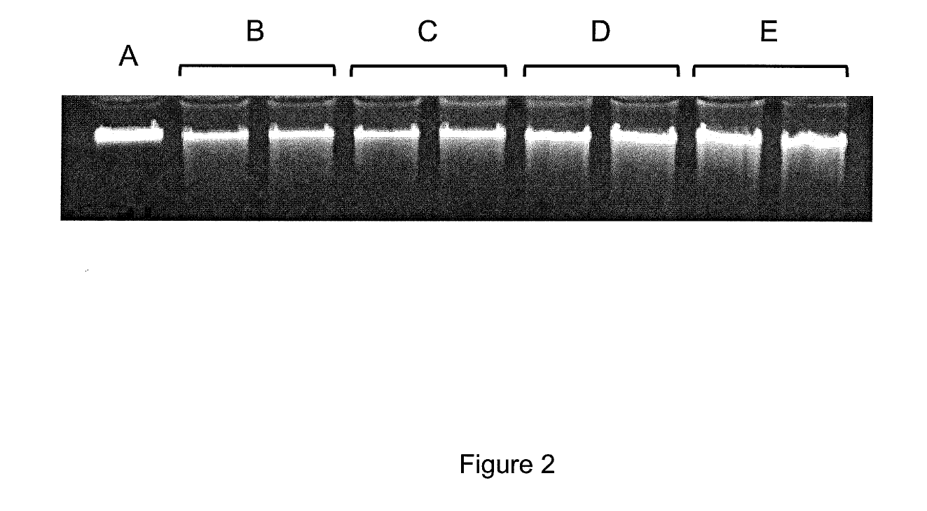

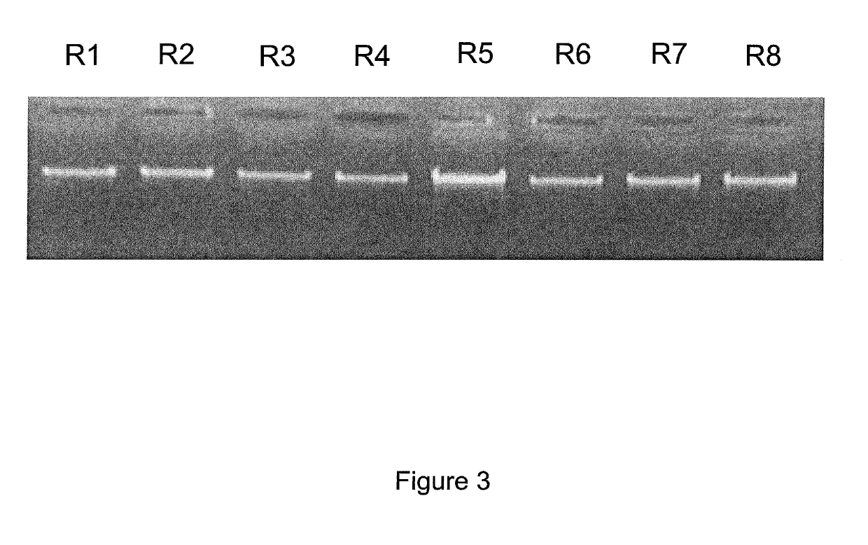

An 8 μL sample of each eluate of isolated DNA was resolved by 0.8% agarose gel electrophoresis (FIG. 3).

### EXAMPLE 6

This example relates to isolation of DNA from plant leaf material from sunflower.

Four punches (6 mm in diameter) were taken from dried sunflower leaf material and ground in 400 μL lysis buffer PN (2.25M guanidinium hydrochloride, 15% Tween-20, 50% 1-propanol) using a ball-milling Instrument (Genogrinder) for 1 minute at 1,750 strokes per second. The resultant sample was incubated for 30 minutes at 60° C., according to the protocol of the Sbeadex Maxi Plant Kit (LGC Genomics GmbH, Cat. No. 41602/41620).

After that incubation, 200 μL lysate was mixed with 400 μL binding buffer PN (2.25M guanidinium hydrochloride, 15% Tween-20, 50% 1-propanol) and 10 μL standard Sbeadex beads solution and shaken for 5 minutes. Following shaking the Sbeadex beads were removed by application of a permanent magnet and the supernatant was removed.

The Sbeadex beads were resuspended in 400 μL wash buffer PN1 (1.5M guanidinium hydrochloride, 10% Tween-20, 20% 1-propanol) and shaken for 5 minutes. The Sbeadex beads were collected by application of a permanent magnet and the supernatant was removed. The Sbeadex beads were then resuspended in 400 μL of a wash buffer containing 0.1 mM calcium chloride in 10 mM TrisHCl (pH 8.0) and shaken for 5 minutes. As before, the Sbeadex beads were collected by application of a permanent magnet.

DNA bound to the Sbeadex beads was eluted by addition of 100 L elution buffer containing 40 mM 2-aminomethylpropanol-2 (pH 10.0) and 0.1 mM ethylene glycol-bis(2-aminoethylether)-N,N,N′,N′-tetraacetic acid (EGTA). Elution was carried out at 50° C. with occasionally shaking of the tube to keep the Sbeadex beads in suspension.

The DNA isolation experiment was repeated a total of eight times and the eluted DNA was measured by UV spectrophotometry (Table 3).

TABLE 3
Eluate DNA concentration following DNA isolation (8 replicates)

| | | | DNA |
|---|---|---|---|
| | | | concentration |
| Replicate number | A_{260/280} ratio | A_{260/230} ratio | (ng/μL) |
| R1 | 1.8 | 0.5 | 13.6 |
| R2 | 1.8 | 0.5 | 11.8 |
| R3 | 1.6 | 0.6 | 25.3 |
| R4 | 1.8 | 0.5 | 17.2 |
| R5 | 1.8 | 0.6 | 43.0 |
| R6 | 1.7 | 0.4 | 29.1 |
| R7 | 1.8 | 0.7 | 40.2 |
| R8 | 1.6 | 0.7 | 52.7 |

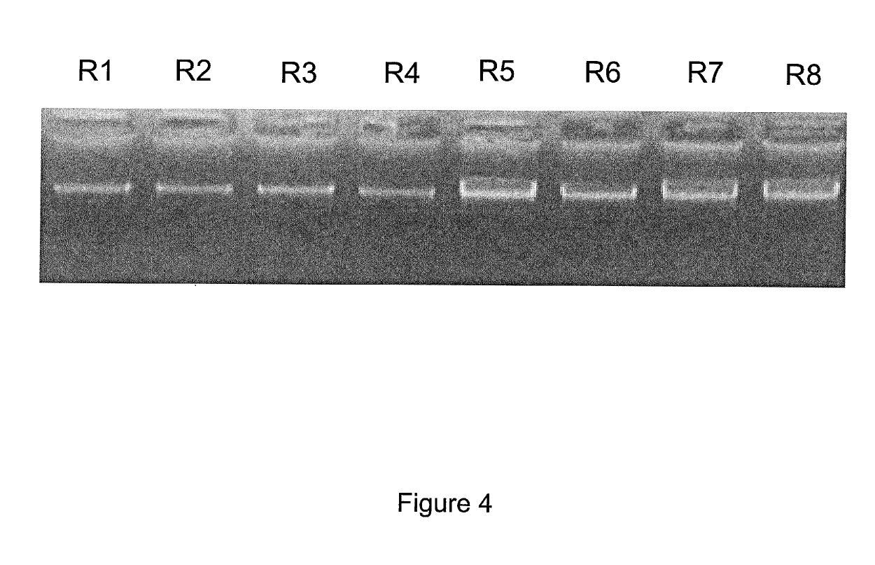

An 8 μL sample of each eluate of isolated DNA was resolved by 0.8% agarose gel electrophoresis (FIG. 4).

1. A method of isolating a nucleic acid wherein the method comprises the steps of:
(a) providing a binding mixture comprising nucleic acid;
(b) contacting the binding mixture with a polar solid support such that nucleic acid adsorbs to the surface of the solid support;
(c) removing unbound binding mixture;
(d) washing the solid support with a wash buffer; and,
(e) adding an elution buffer and removing nucleic acid from the solid support.

2. The method of claim 1 wherein the wash buffer comprises bivalent cations in aqueous solution.

3. The method of claim 2 wherein the bivalent cations are from alkaline earth metals from Group II of the periodic table.

4. The method of claim 2 or claim 3 wherein the bivalent cations are in the form of their chlorides.

5. The method of any of claims 2-4 wherein the bivalent cations are calcium ions or barium ions.

6. The method of claim 5 wherein the bivalent cations are calcium ions.

7. The method of any of claims 2-6 wherein the bivalent cations are at a concentration between 0.1 mM and 10 mM, preferably between 1 mM and 5 mM.

8. The method of any of claims 2-7 wherein the nucleic acid is removed from the surface of the solid support by complexing bivalent cations.

9. The method of claim 8 wherein the elution buffer comprises a chelating agent.

10. The method of claim 9 wherein the chelating agent complexes the bivalent cations provided in the wash buffer, promoting removal of the nucleic acid from the surface of the solid support.

11. The method of claim 9 or claim 10 wherein the chelating agent is EGTA, EDTA, EDDS, MGDA, IDS, polyaspartic acid, or GLDA, preferably EGTA.

12. The method of any of claims 8-11 wherein the chelating agent is at a concentration between 0.1 mM and 5 mM, preferably at a concentration between 0.1 mM and 1 mM.

13. The method of any of claims 8-12 wherein the elution buffer has a pH value of between pH 7.0 and pH 10.0, preferably a pH value of between pH 7.0 and pH 9.0.

14. The method of any of claims 1-7 wherein the nucleic acid is removed from the surface of the solid support by raising the pH of the solid support to alkaline conditions.

15. The method of claim 13 wherein the elution buffer has a pH value between pH 7.1 and pH 10.0, preferably between pH 8.0 and pH 10.0, more preferably between pH 9.0 and pH 10.0.

16. The method of any of claims 1-7 wherein the nucleic acid is removed from the surface of the solid support by raising the temperature of the solid support, and complexing bivalent cations as defined in any of claims 8-13, and/or raising the pH of the solid support to alkaline conditions as defined in claim 14 or claim 15.

17. The method of claim 16 wherein the temperature is raised to at least 30° C., preferably wherein the temperature is raised to between 30° C. and 75° C.

18. The method of any of the preceding claims wherein the surface of the solid support binds bivalent cations.

19. The method of claim 18, wherein the binding of bivalent cations to the surface of the solid support leads to adsorption of nucleic acid to the surface of the solid support.

20. The method of any of the preceding claims wherein the surface of the solid support comprises weak organic acids.

21. The method of claim 20 wherein the weak organic acids are homo- or hetero-polymers.

22. The method of claim 21 wherein the weak organic acid polymer is selected from the list consisting of poly-acrylic acid, poly-phosphonic acid, poly-methacrylic acid, a hetero-polymer of methacrylic acid and maleic acid, and a hetero-polymer of acrylic acid, methacrylic acid and maleic acid.

23. The method of claim 20 wherein the weak organic acids are selected from the list consisting of phosphonic acids, aliphatic carboxylic acids, and aromatic carboxylic acids.

24. The method of any of the preceding claims wherein the binding mixture comprises a binding buffer.

25. The method of claim 24 wherein the binding buffer comprises an organic solvent that is miscible with water, and/or a chaotropic agent, and/or a detergent.

26. The method of claim 25 wherein the binding buffer comprises at least two of: an organic solvent that is miscible with water; a chaotropic agent; and a detergent.

27. The method of claim 25 or claim 26 wherein the volume/volume percentage of organic solvent in the binding buffer Is at least 5%, preferably between 5% and 50%, more preferably between 20% and 50%, yet more preferably between 30% and 50%, most preferably between 40% and 50%.

28. The method of any of claims 25-27 wherein the concentration of chaotropic agent in the binding buffer is at least 0.5M, preferably between 0.5M and 3M, more preferably between 0.5M and 1.5M.

29. The method of any of claims 25-28 wherein the volume/volume percentage of detergent in the binding buffer is at least 0.5%, preferably between 5% and 20%, more preferably between 7% and 15%, most preferably between 8% and 12%.

30. The method of any of claims 25-29 wherein the organic solvent is an alcohol, preferably a low molecular weight alcohol, more preferably ethanol, 1-propanol, or propan-2-ol.

31. The method of any of claims 25-30 wherein the chaotropic agent is a guanidinium salt, preferably guanidinium thiocyanate or guanidinium, hydrochloride, more preferably guanidinium hydrochloride.

32. The method of any of claims 25-31 wherein the detergent is an ionic detergent or a non-ionic detergent.

33. The method of any of claims 25-32 wherein the ionic detergent is a polyethylene glycol (PEG)-based detergent.

34. The method of any of claims 25-33 wherein the non-ionic detergent comprises a weak organic acid group.

35. The method of any of the preceding claims wherein the solid support comprises microparticles.

36. The method of claim 35 wherein the microparticles have superparamagnetic properties.

37. The method of claim 35 or claim 36 wherein the microparticles have a diameter of at least 1 μm, preferably between 1 μm and 50 μm, more preferably between 1 μm and 20 μm.

38. The method of any of the preceding claims wherein the isolated nucleic acid is DNA, RNA, PNA, GNA, TNA, or LNA, preferably DNA or RNA.

39. The method of any of the preceding claims wherein the isolated nucleic acid is at least 20 nucleotides in length.

40. The method of any of the previous claims wherein the nucleic acid is isolated from a starting sample.

41. The method of claim 40 wherein, prior to the isolation of the nucleic acid, the starting sample is treated using one or more of: chemical treatment, enzymatic treatment, and/or mechanical treatment.

42. The method of claim 40 or claim 41 wherein the starting sample comprises laboratory contaminants.

43. The method of claim 42 wherein the laboratory contaminants comprise Polymerase Chain Reaction (PCR) reagents, restriction enzyme digest reagents, in vitro reagent systems for modifying and/or processing nucleic acids, acrylamide gel, or agarose gel.

44. The method of claim 40 or claim 41 wherein the starting sample comprises biological material.

45. The method of claim 44 wherein the biological material comprises eukaryotic or prokaryotic cells.

46. The method of claim 45 wherein the cells are animal cells, plant cells, fungal cells, bacterial cells, archaeal cells, or protozoan cells.

47. The method of any of claims 44-46 wherein the biological material is a bodily fluid or solid biological material from an animal.

48. The method of claim 47 wherein the bodily fluid or solid biological material is selected from: blood, plasma, serum, urine, faeces, saliva, semen, nail, hair, or tissue.

49. The method of claim 40 wherein the starting sample is material obtained for forensic analysis.

50. The method claim 49 wherein the material comprises saliva, blood, urine, faeces, semen, sweat, tears, hair, nail, or any tissue.

51. The method of any of the preceding claims wherein prior to removal, the nucleic acid remains adsorbed to the surface of the solid support even when the chemical conditions which promote adsorption to the surface of the solid support are no longer present.

52. The method of any preceding claim wherein the elution buffer is an aqueous elution buffer.

53. The method of claim 53 wherein the buffer comprises tris(hydroxymethyl)aminomethane.

54. The method of claim 53 wherein the tris(hydroxymethyl)aminomethane is at a concentration between 1 mM and 50 mM, preferably between 1 mM and 20 mM, most preferably between 5 mM and 15 mM.

55. A kit comprising:
(a) a polar solid support as defined in any of claims 1 and 35-37;
(b) a binding buffer as defined in any of claims 1 and 24-34;
(c) a wash buffer as defined in any of claims 1-7; and,
(d) instructions for use.

56. The kit of claim 55 wherein the kit further comprises:
(f) an elution buffer as defined in any of claims 1, 8-16, and 52-54.
